(19) United States
(12) Patent Application Publication
(10) Pub. No.: US 2024/0237992 A1
(43) Pub. Date: Jul. 18, 2024

(54) QUICK CONNECT SAW ATTACHMENT FOR SURGICAL INSTRUMENTS

(71) Applicant: Arthrex, Inc., Naples, FL (US)

(72) Inventors: Giuseppe Lombardo, Trinity, FL (US); Joseph A. Fritz, Seminole, FL (US); Andrew P. Muser, Clarkesville, GA (US)

(73) Assignee: Arthrex, Inc, Naples, FL (US)

(21) Appl. No.: 18/406,409

(22) Filed: Jan. 8, 2024

Related U.S. Application Data
(60) Provisional application No. 63/438,656, filed on Jan. 12, 2023.

(51) Int. Cl. A61B 17/14

(52) U.S. Cl. CPC A61B 17/14; A61B 2017/00473

(57) ABSTRACT

A blade-mounting apparatus for a surgical tool includes an attachment head including a mounting channel with a plurality of opposing sides and a base. Opposing slots are formed in each of the opposing sides and aligned across the mounting channel forming a blade-receiving opening extending therebetween and aligned with a receiving axis. A locking detent is disposed within the mounting channel and has an engagement surface including a plurality of protrusions with a first protrusion proximal to the receiving opening and a second protrusion distal to the receiving opening. The second protrusion is elongated transverse to the receiving axis and tapered from the engagement surface to a distal extent.

### CROSS-REFERENCE TO RELATED APPLICATIONS

This application claims priority under 35 U.S.C. § 119(e) and the benefit of U.S. Provisional Application No. 63/438,656 entitled Quick Connect Saw Attachment for Surgical Instruments, filed on Jan. 12, 2023, by Giuseppe Lombardo, et al., the entire disclosure of which is incorporated herein by reference.

### BACKGROUND

The present disclosure generally relates to an apparatus for the interchangeable attachment of accessories for surgical instruments. More particularly, the disclosure relates to a connection interface and complimentary apparatus configured for the installation and removal of attachable accessories or consumables in connection with a surgical tool.

### SUMMARY

During surgical procedures, saw blades and similar wear components may necessarily be rapidly exchanged to prevent downtime and related procedural delays. Accordingly, the disclosure provides for a quick-connect blade or accessory mounting apparatus that may assist in the rapid replacement and exchange of blades and similar accessories. In various implementations, the blade-mounting apparatus comprises an attachment head that works in combination with a locking detent to engage and secure a thickness of a blade within a mounting channel. In some examples, the disclosure may provide for the locking detent and attachment head to accommodate blades and similar implements having a range of thicknesses, such that the mounting apparatus may be flexibly applied to effectively secure a wide variety of blades and consumable accessories. In addition to securing the blade and providing flexibility to accommodate a variety of thicknesses, the disclosure may further provide for an easily assembled and accessible mounting structure that may be readily accessed and disassembled to ensure effective sterilization between uses.

In some implementations, the attachment head of the mounting apparatus may form a mounting channel including opposing sides and a base. Opposing slots may be formed in the opposing sides and aligned across the mounting channel to form a blade-receiving opening aligned with a receiving axis. A locking detent may be disposed within the mounting channel and comprise an engaging surface including a plurality of protrusions. The protrusions may include a first protrusion proximal to the receiving opening and a second protrusion distal from the receiving opening. The second protrusion may be elongated transverse to the receiving axis and taper as it extends away from the engagement surface to a distal extent. In various implementations, the proportions and/or positions of the first and second protrusions relative to corresponding first and second blade apertures may vary to ensure that the blade-mounting apparatus is capable of securing blades having a wide range of thicknesses.

In some implementations, one or more characteristics of the blade apparatus may vary to accommodate and ensure that the locking detent and attachment head can effectively secure blades over a range of blade thicknesses. The surgical blade may incorporate an elongated rectangular mating profile defining a blade width and a blade thickness extending along a longitudinal axis of the blade. The elongated rectangular mating profile may be configured to engage a blade-receiving opening formed by a blade-attachment assembly. An acting end may be disposed at a first end portion of the blade apparatus, and a mounting interface may be disposed at a second end portion of the blade apparatus. The mounting interface may include a plurality of mating apertures configured to receive a plurality of mating protrusions or complimentary protrusions formed through the surgical blade along a mating axis perpendicular to the longitudinal axis.

The plurality of apertures may include a fore aperture formed at a first longitudinal position comprising a first length and a first width. The fore aperture may form a first receiving perimeter wall configured to receive the first protrusion of the mating protrusions. An aft aperture may be formed at a second longitudinal position comprising a second length and a second width. The aft aperture may form a second receiving aperture wall configured to receive the second protrusion of the mating protrusions. In various implementations, the fore aperture and the aft aperture may include one or more variable locating dimensions defined by at least one of the first longitudinal position, the first length, and the second length. The variable locating dimensions may vary to ensure that the blade-attachment assembly may effectively secure blades having a range of blade thicknesses.

In yet another implementation, a method of mounting a blade to a blade-mounting apparatus of a surgical tool may include guiding a first end of the blade along a receiving axis of a receiving opening formed between opposing slots aligned over a mounting channel. The method may further include sliding the first end of the blade along a ramp of the fore protrusion of a locking detent by displacing the locking detent from a clearance portion of the receiving opening. With the blade located within the clearance portion, the blade may further be guided, such that an aft aperture of the blade passes over and beyond the fore protrusion. The unencumbered passage of the blade over the fore protrusion may be the result of a receiving dimension of the aft aperture of the blade being insufficient to receive the fore protrusion. The method may further include aligning a fore aperture of the blade over the fore protrusion concurrently with aligning the aft aperture of the blade over an aft protrusion of the locking detent. Once aligned, the blade may be locked into place or mounted in position by extending the locking detent into the clearance portion of the receiving opening. In the mounted position, the protrusions of the locking detent may engage the apertures of the blade, thereby securing the blade to the blade-mounting apparatus of the surgical tool.

These and other features, objects and advantages of the present disclosure will become apparent upon reading the following description thereof together with reference to the accompanying drawings.

### BRIEF DESCRIPTION OF THE DRAWINGS

FIG. 1 is a projected view of a surgical tool demonstrating a blade-mounting apparatus;

FIG. 2 is a projected view of a tool head demonstrating a blade-mounting apparatus and a compatible blade including a mounting interface;

FIG. 3 is a side cross-sectional view of a tool head demonstrating a blade-mounting apparatus;

FIG. 4A is a projected view of an attachment head of a blade-mounting apparatus;

FIG. 4B is a side cross-sectional view of the attachment head of the blade-mounting apparatus;

FIG. 5A is a projected view of a locking detent and guide shaft of a blade-mounting apparatus;

FIG. 5B is a side cross-sectional view of the locking detent of the blade-mounting apparatus;

FIG. 6A is a projected view of a blade or tool accessory compatible with the blade-mounting apparatus demonstrating a mounting interface;

FIG. 6B is a top view of a blade compatible with the blade-mounting interface;

FIG. 7A is a detailed side cross-sectional view of a blade-mounting apparatus demonstrating a blade-mounting method;

FIG. 7B is a detailed side cross-sectional view of a blade-mounting apparatus demonstrating a blade-mounting method;

FIG. 7C is a detailed side cross-sectional view of a blade-mounting apparatus demonstrating a blade-mounting method; and

FIG. 7D is a detailed side cross-sectional view of a blade-mounting apparatus demonstrating a blade unloading method.

### DETAILED DESCRIPTION

In the following description, reference is made to the accompanying drawings, which show specific implementations that may be practiced. Wherever possible, the same reference numbers will be used throughout the drawings to refer to the same or like parts. It is to be understood that other implementations may be utilized, and structural and functional changes may be made without departing from the scope of this disclosure.

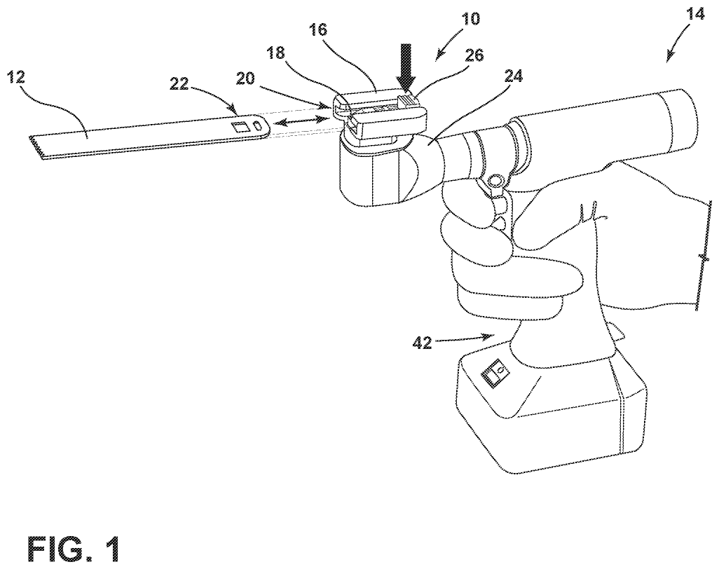

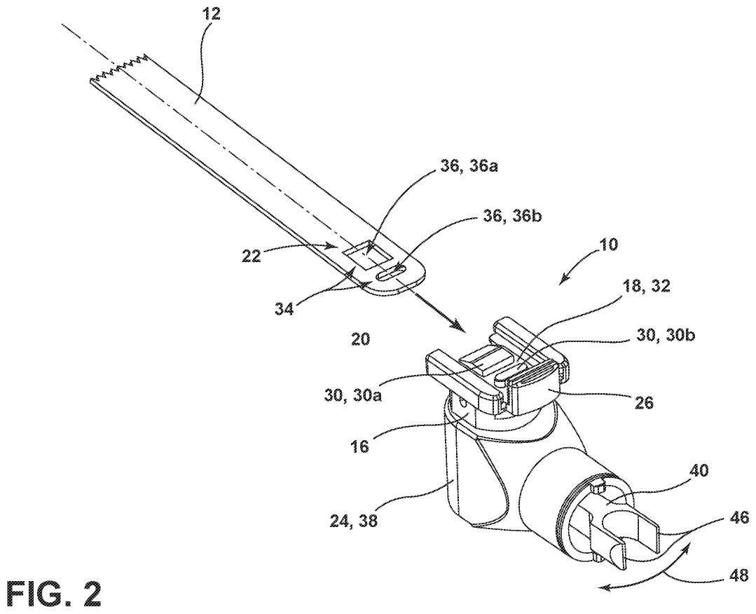

Referring to FIGS. 1 and 2, the disclosure generally provides for a blade-mounting apparatus 10 configured to securely mount and retain a blade 12 or similar consumable accessory for use with a surgical tool 14. In various implementations, the mounting apparatus 10 may include an attachment head 16 and locking detent 18 forming a blade-retention slot or opening 20 configured to receive the blade 12. Once positioned within the blade-retention slot or opening 20, the locking detent 18 may engage a mounting interface 22 of the blade 12, thereby securing the blade 12 to an actuating head 24 of the tool 14. Once the use of the blade 12 is completed, the mounting apparatus 10 may additionally provide for a simple ejection or removal process by depressing a release tab 26 disengaging the locking detent 18 from the mounting interface 22 of the blade 12. In this way, the blade-mounting apparatus 10 may provide for an intuitive solution for mounting and ejecting the surgical blade 12 or similar consumable accessories.

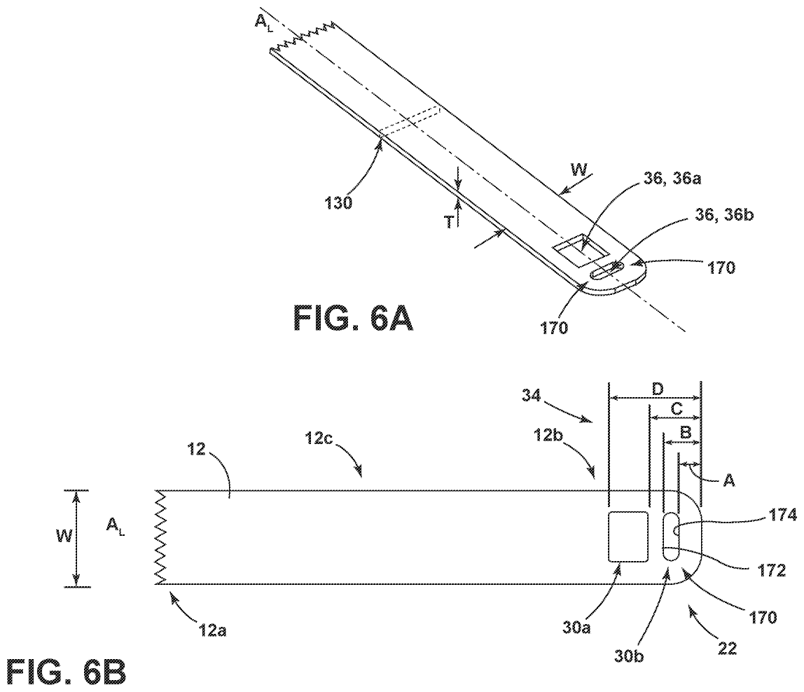

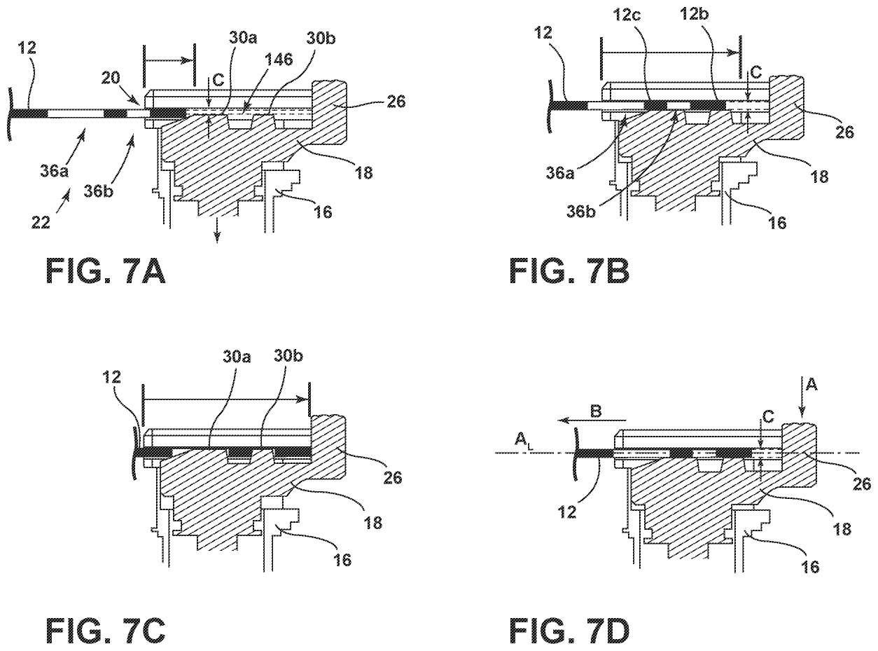

In various implementations, the mounting apparatus 10 may be configured to securely engage tool accessories having a variety of thicknesses. The engagement of such tool accessories, exemplified herein as the blade 12, may be provided by the interaction of a plurality of protrusions 30 (FIG. 2) extending from an engagement surface 32 of the locking detent 18. The protrusions 30 may include a variety of features discussed in further detail throughout the application and specifically in reference to FIGS. 6A and 6B. The engagement features of the protrusions 30 may ensure that the mounting interface 22 of the blade 12 is secured to the attachment head 16 of the mounting apparatus 10. One or more dimensions of the mounting interface 22 of each of the blades 12 may vary depending on a thickness of the blade 12. For example, the mounting interface 22 may include one or more variable locating dimensions 34 of a plurality of apertures 36 that vary to accommodate the receipt of the protrusions 30 of the locking detent 18. Further detailed description of exemplary variable locating dimensions 34 and features of the mounting interface 22 implemented based on the thickness of the blade 12 are discussed throughout the detailed description and particularly in reference to FIGS. 7A and 7B. Accordingly, the disclosure may provide for the mounting apparatus 10 to securely engage complimentary features of the blades 12 or accessories having various thicknesses T to ensure effective operation of the surgical tool 14 for a variety of applications.

Referring now to FIGS. 1, 2, and 3, the attachment head 16 of the mounting apparatus 10 may engage the actuating head 24 via a drive link 40. The drive link 40 may be enclosed within a body 38 of the actuating head 24 and engage a motor drive of a handpiece 42 of the surgical tool 14 via a mounting collar 44. In operation, prongs 46 of the drive link 40 may engage a bearing assembly (not shown) of the motor drive of the handpiece 42 and generate an oscillating motion 48 at the drive link 40. In this way, the motor drive may selectively actuate the oscillating motion 48 and generate corresponding oscillations in the attachment head 16 of the mounting apparatus 10. Though the drive link 40 and actuating head 24 are described in reference to the specific examples demonstrated, it shall be understood that the mounting apparatus 10 may be implemented in combination with a variety of linear or rotational oscillating or reciprocating actuation linkages and corresponding surgical tools.

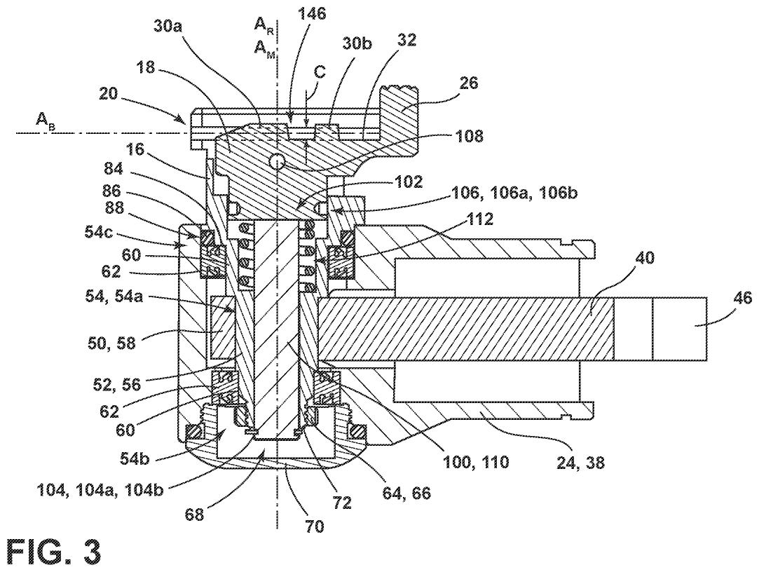

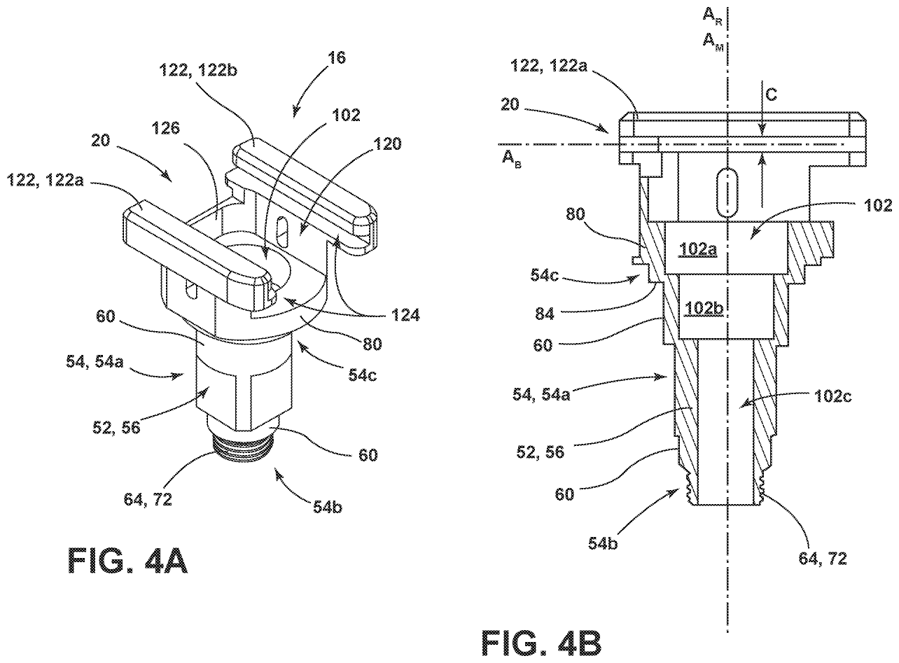

Referring now to FIGS. 3, 4A, and 4B, a drive interface between the actuating head 24 and the attachment head 16 of the mounting apparatus 10 is demonstrated in further detail. In the example shown, the drive link 40 extends through the body 38 of the actuating head 24. As previously discussed, the prongs 46 may engage a motor drive interface of the handpiece 42 on a first end and a drive coupler or drive socket 50 positioned on an opposing, acting end of the drive link 40. The drive socket 50 may engage a drive head 52 formed by a shank 54 of the attachment head 16 that extends into the body 38 of the actuating head 24. The drive head 52 may form an engagement profile 56 (for example, a square, hexagonal, star, or similar profile shape) configured to engage and mesh with a complimentary receiving profile 58 formed by the drive socket 50 of the drive link 40. In the exemplary embodiment shown, the engagement profile 56 is a square drive having four equally proportioned and spaced sides extending about the perimeter of an intermediate portion 54a of the shank 54. In this configuration, the oscillating motion 48 of the drive link 40 may be efficiently communicated to generate a rotational motion of the attachment head 16 about a rotational axis Aᵣ.

The shank 54 of the attachment head 16 may correspond to a rigid body forming or interconnected with the drive head 52 that extends into the body 38 of the actuating head 24. In operation, the drive head 52 engages the drive socket 50 to communicate the oscillating motion 48 to drive or rotate the mounting apparatus 10 about the rotational axis Aᵣ. On opposing ends of the drive head 52, the shank 54 may further form rotational support surfaces 60 that may be configured to engage rotational supports 62. In various implementations, the rotational supports 62 may correspond to bearing assemblies, bushings or supports connected to an interior wall formed within the body 38 of the actuating head 24. In this configuration, the rotation of the shank 54 within the actuating head 24 may be aligned with the rotational axis A_{R} and friction associated with the oscillating motion 48 may be mitigated.

On opposite ends of the rotational supports 62 (e.g., opposite longitudinal extents of the shank 54), the shank 54 may be axially confined and positioned in connection with the body 38 of the actuating head 24. In this configuration, the shank 54 may secure the mounting apparatus 10 in mechanical attachment with the actuating head 24 while allowing controlled motion of the mounting apparatus 10 about the rotational axis Aᵣ. On a first side, the shank 54 may comprise an attachment end 54b opposite the blade-receiving opening 20 formed by the attachment head 16 and the locking detent 18. On the attachment end 54b, the shank 54 may comprise or form a retaining feature 64 (e.g., a threaded end portion, annular groove, etc.). The retaining feature 64 may engage a retaining ring 66 (FIG. 3) positioning the shank 54 and the attachment head 16 relative to the actuating head 24 on the first side along the rotational axis A_{R}.

As best illustrated in FIG. 3, the retaining feature 64 may extend into an assembly pocket 68 that may be selectively enclosed by an end cap 70. In the example shown, the retaining feature 67 may correspond to a threaded end portion 72 of the shank 54. Additionally, the retaining ring 66 may correspond to a locking fastener (e.g., a lock nut). In this configuration, the retaining ring 66 may be secured against the rotational support 62 (e.g., a bearing assembly) to axially position the shank 54 of the mounting apparatus 10 to the actuating head 24 along the rotational axis A_{R}. The removal of the end cap 70 may provide access to readily disassemble the mounting apparatus 10 from the actuating head 24 for maintenance or cleaning.

On a second side of the rotational supports 62, opposite the attachment end portion 54b, the shank 54 may be in connection with or form a base 80 or support base of the attachment head 16. On the second side, the shank 54 may be positioned along the rotational axis A_{R} via a stepped end portion 54c or stepped shank diameter. As shown the shank 54 may be drawn in tension between a mounting ledge 84 of the stepped end portion 54c and the retaining ring 66 at the attachment end portion 54b. Additionally, proximate to the mounting ledge 84, a head seal 86 may be disposed in a perimeter recess 88 formed between the stepped perimeter wall 82 and the body 38 of the actuating head 24. The head seal 86 may correspond to a ring seal disposed in an annular opening formed by the perimeter recess 88 about a perimeter of the shank 54. In this configuration, the drive head 52 and the drive socket 50 may be sealed within the actuating head 24 to prevent infiltration of fluids or debris into the drive link 40 and drive assembly while allowing rotation of the shank 54 and the mounting apparatus 10 about the rotational axis A_{R}.

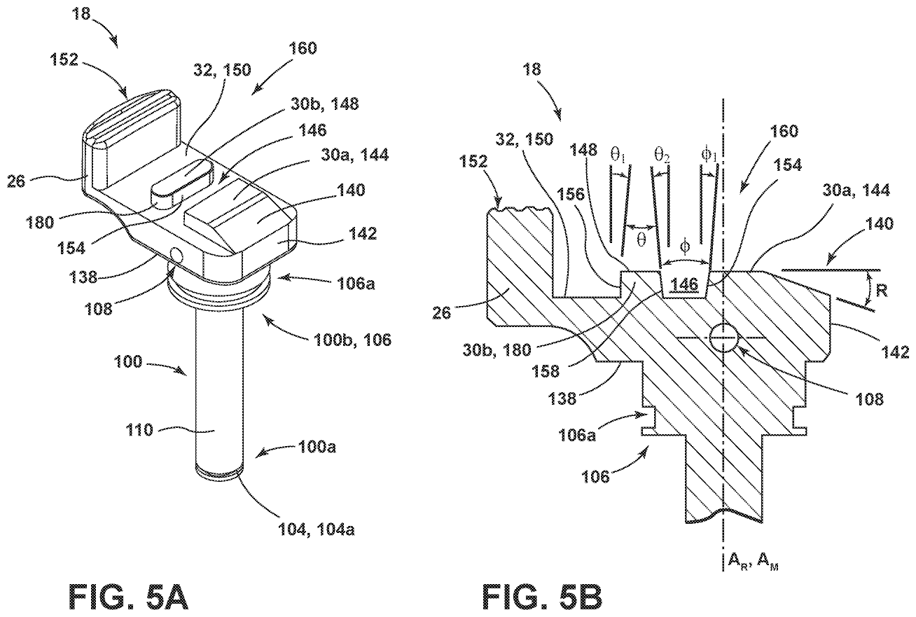

With the mounting apparatus 10 engaged with the drive link 40 of the actuating head 24, the oscillating motion 48 of the drive link 40 may result in corresponding oscillations in the mounting apparatus 10 and any connected blades 12 or tool accessories. Referring now to FIGS. 2-5, an exemplary operation of the interaction between the attachment head 16 and the locking detent 18 to secure the blade 12 is discussed in further detail. As best demonstrated in FIG. 3, the locking detent 18 may be secured to the attachment head 16 via a detent shaft 100 extending into and captured within a bore 102 centrally formed within the shank 54 along the rotational axis A_{R}. As shown in FIG. 5A, the detent shaft 100 may comprise a retention feature 104 disposed on a first side 100a and an intermediate stepped portion 106 on a second side 100b proximate to the locking detent 18. Though not demonstrated in FIG. 6A or 6B, the detent shaft 100 may further extend into a body of the locking detent 18 and be secured via a press-fit rod or fastener within a mating aperture 108 extending through the locking detent 18 and the detent shaft 100.

As shown in FIG. 3, the retention feature 104 may correspond to an annular groove 104a configured to receive a retention ring 104b or retention pin, thereby securing a stem 110 of the detent shaft 100 within the bore 102 of the shank 54 and against the attachment end portion 54b. Opposite the retention feature 104, on the second side 100b, the intermediate stepped portion 106 of the detent shaft 100 is disposed within a first interior stepped portion 102a of the bore 102. In this configuration, the intermediate or exterior stepped portion 106 of the detent shaft 100 may slide within the first interior stepped portion 102a of the bore 102 along the longitudinal axis of the shank 54 and the rotational axis A_{R}.

With the detent shaft 100 and the locking detent 18 bound to the shank 54 of the attachment head 16 within the bore 102, a travel of the locking detent 18 may be defined by a travel length of the intermediate stepped portion 106 within the first interior stepped portion 102a of the bore 102. In an assembled configuration, the locking detent 18 may be maintained in an extended position by a spring mechanism 112 configured to apply a spring force positioning the locking detent 18 away from the bore 102 (vertically as depicted). In an unloaded state wherein a blade 12 is not engaged with the mounting apparatus 10, the retention feature 104 may retain the locking detent 18 and detent shaft 100 in connection with the shank 54. As demonstrated in FIG. 3, the spring mechanism 112 may correspond to a coil spring disposed within the first interior stepped portion 102a and a second interior stepped portion 102b (FIG. 4B) of the bore 102 between the intermediate or exterior stepped portion 106 of the detent shaft 100 and a third interior stepped portion 102c of the bore 102. In this configuration, the spring mechanism 112 may be effectively captured in compression between the exterior stepped portion 106 of the detent shaft 100 on the second side 100b and a ledge formed between second interior stepped portion 102b and the third interior stepped portion 102c. Accordingly, a force applied along the axis of rotation A_{R} may compress the spring mechanism 112 and create a clearance region C (FIGS. 3 and 4B) within the blade-receiving opening 20 as a result of a displacement of the locking detent 18 along the axis of rotation A_{R}.

As previously discussed, a length of the first interior stepped portion 102a formed by the bore 102 within the shank 54 may define a travel distance of the detent shaft 100 and the locking detent 18 as a result of the compression of the spring mechanism 112 along the axis of rotation A_{R}. In order to effectively seal the bore 102 from contamination and associated wear, one or more seals may be incorporated in the mating configuration between the base 80 and shank 54 of the attachment head 16 and the detent shaft 100. In various implementations, the intermediate or exterior stepped portion 106 may comprise an annular groove 106a configured to receive a seal 106b. In this configuration, the exterior stepped portion 106 may traverse the first interior stepped portion 102a and create a perimeter seal about the annular groove 106a to prevent contaminants from entering the bore 102.

Referring now to FIGS. 3, 4A, 4B, 5A, and 5B, the exemplary operation of the mounting apparatus is discussed in reference to the engagement features and corresponding operation of the attachment head 16 and the locking detent 18. As best demonstrated in FIGS. 4A and 4B, the attachment head 16 may form a mounting channel 120 comprising opposing sides 122 separated across and extending from the support base 80. Opposing slots 124 may be formed in each of the opposing side 122 and aligned across the mounting channel 120 forming the blade-receiving opening 20. The blade-receiving opening 20 may extend across the mounting channel 120 and define a blade-receiving axis A_{B} positioned centrally between the opposing sides 122 and the opposing slots 124. As later discussed in reference to FIG. 7, a longitudinal axis A_{L} of the blade 12 may be aligned with the blade-receiving axis A_{B} during a mounting operation, such that an elongated rectangular mating profile 130 of the blade 12 is aligned between the opposing sides 122 and within the opposing slots 124. As demonstrated in the example shown in FIG. 4A, the opposing sides 122 may be partially connected between the support base 80 and the opposing slots 124 along the receiving end 122a of the attachment head 16. The connected portion extending along the receiving end 122a may be referred to as a lateral wall 126, which may provide for reinforcement of the opposing sides 122 as well as to prevent debris from entering the mounting channel 120. Further, as best illustrated in FIG. 3, the lateral wall 126 may provide a guide surface for the locking detent 18 to slide along the rotational axis A_{R}.

Still referring to FIG. 4, in some implementations, the opposing slots 124 may extend unincumbered from a receiving end 122a to an open terminal end portion 122b of the opposing sides 122. In this configuration, the receiving end 122a of the opposing sides 122 and the corresponding slots 124 may be configured to receive the elongated mating profile 130 of the blade 12 within the receiving opening 20 and allow the length of the blade 12 along the longitudinal axis A_{L} to slide through the opposing slots 124 to and beyond the open terminal end portion 122b of the attachment head 16. The open receiving end 122a and opposing terminal end portion 122b of the opposing slots 124 formed through the attachment head 16 may provide for the interior of the opposing slots 124 to be easily cleaned and purged of contaminants after use. It shall be understood that the ease of cleaning and reduction of material buildup associated with surgical equipment, such as the surgical tool 14, may be particularly beneficial for sterilization and sanitation between uses.

As shown in FIGS. 5A and 5B, the locking detent 18 may comprise the protrusions 30a, 30b extending from the engagement surface 32. As shown in FIGS. 1 and 2, the locking detent 18 may be disposed within the mounting channel 120 between the opposing sides 122. As previously discussed, the locking detent 18 may be connected to the second side 100b of the detent shaft 100 at a connection surface 138 adjacent to the exterior stepped portion 106. In this configuration, the locking detent 18 may slide up and down as depicted in FIG. 3, along the rotational axis A_{R}, by sliding the detent shaft 100 within the bore 102. In response to the sliding of the detent shaft 100, the clearance region C may be selectively revealed to receive or release the blade 12 within the blade-receiving opening 20.

Still referring to FIGS. 5A and 5B, the fore protrusion 30a may be positioned proximally to the receiving end 122a relative to the aft protrusion 30b. The fore protrusion 30a may comprise a ramp portion 140 extending at a ramp angle R from a fore end 142 of the locking detent 18. As shown, the ramp angle R may be approximately 20°, however, in various implementations the ramp angle R may vary from approximately 10° to 40°. The ramp portion 140, as shown, may extend from the fore end 142 to an elongated protrusion surface 144, which may correspond to a plateau or tabletop proximate to the fore end 142. In this configuration, the elongated mating profile 130 of the blade 12 may enter the blade-receiving opening 20, contact the ramp portion 140 between the opposing slots 124, and compress the spring mechanism 112, thereby exposing the clearance region C to receive the blade 12 along the ramp portion 140 and the elongated protrusion surface 144.

The aft protrusion 30b may protrude outward from the engagement surface 32 forming a transverse recess 146 between the fore protrusion 30a and the aft protrusion 30b. The aft protrusion 30b may form a narrow protrusion surface 148 relative to the elongated protrusion surface 144 of the fore protrusion 30a. Still moving away from the fore end 142 of the locking detent 18, the engagement surface 32 may extend to an aft end portion 150 from which the release tab 26 may extend to an interface surface 152. In the assembled configuration demonstrated in each of FIGS. 1, 2, and 3, the interface surface 152 of the release tab 26 is demonstrated extending or exposed beyond the terminal end portion 122b of the opposing sides 122 along the blade-receiving axis A_{B}, as well as above a height of the opposing sides 122 along the rotational axis A_{R}. In this configuration, the interface surface 152 of the release tab 26 may be readily accessible by a user to depress the locking detent 18 to expose the clearance region C to release or receive the blade 12.

Still referring primarily to FIGS. 5A and 5B, the transverse recess 146 may be formed between first tapered opposing sides 154 forming a locking recess having an interior angle ¢, which may vary from approximately 5° to 45°. Adjacent to the transverse recess 146, forming an aft side of the tapered opposing sides 154, the aft protrusion 30b may extend from the engagement surface 32 and form second tapered opposing sides 156. In this configuration, the first tapered opposing sides 154 may share a common side portion 158 with the second tapered opposing sides 156. The second tapered opposing sides of the aft protrusion 30b may extend from the engagement surface 32 at a taper angle θ, which may similarly range from approximately 5° to 45°. The combination of the first tapered opposing sides 154 formed by the transverse recess 146 and the second tapered opposing sides 156 formed by the aft protrusion 30b may form a locking interface 160 in combination with the ramp portion 140 to engage the mounting interface 22 of the blade 12 as further discussed in reference to FIGS. 6 and 7.

As demonstrated in FIG. 5B, the taper angle θ may be formed by a first taper angle θ₁ and a second taper angle θ₂ of the second tapered opposing sides 156. The interior angle ϕ may be formed by the first tapered opposing sides 154 by a first interior angle ϕ₁ and a second interior angle θ₂. As shall be understood, the angle associated with the common side portion 158, is the common shared angle denoted as the second tapered angle θ₂. In various implementations, the angles θ₁ and θ₂ and ϕ₁ may vary from approximately 2.5° to 20°. As demonstrated in the example shown in FIG. 5B, the angles θ₁ and θ₂ and ϕ₁ are each approximately 5°, such that the locking interface 160 is defined by a profile with an appearance similar to a square wave having a side angle corresponding to the angles θ₁ and θ₂ and ϕ₁. Though demonstrated as having similar angles, in various implementations, each of the angles θ₁ and θ₂ and ϕ₁ may vary individually to suit a desired application.

Referring now to FIGS. 6A and 6B, further details regarding the blade 12 and the variable locating dimensions 34 are described to clearly demonstrate the interaction between the mounting interface 22 and the locking interface 160. As shown, the blade or blade apparatus 12 may extend along the elongated mating profile 130 from a first end portion 12a to a second end portion 12b. The elongated side of the rectangular mating profile 130 may define a lateral width or blade width W extending perpendicular to a blade thickness T. The blade-receiving opening 20 of the mounting apparatus 10 may be proportioned to provide a generous clearance fit to accommodate the blade thickness T and width W in various implementations. The first end 12a of the blade 12 may correspond to an acting end. The second end 12b of the blade 12 may comprise the mounting interface 22 formed by the apertures 36. As previously discussed, the apertures 36 may be proportioned and configured to receive the protrusions 30 of the locking detent 18 along a mating axis A_{M} aligned with the axis of rotation A_{R} and perpendicular to the longitudinal axis A_{L}. As discussed in the following detailed description, one or more dimensions associated with the position and/or proportions of the apertures 36 may vary based on the blade thickness T to ensure that each of a series or group of compatible blades may be securely mounted and fit the protrusions 30 of the mounting apparatus 10.

As best demonstrated in FIG. 6B, exemplary dimensions of a fore aperture 36a and an aft apertures 36b forming the mounting interface 22 are denoted by dimensions A, B, C, and D. The exemplary values corresponding to the dimensions A, B, C, and D are demonstrated in Table 1 for the aft aperture 36b and Table 2 for the fore aperture 36a. In the examples shown, an aperture width of each of the apertures 36 may be common and omitted from the exemplary dimensions. However, in various implementations, the width of the fore aperture 36a or the aft apertures 36b may vary to accommodate the proportions of the locking interface 160 formed by the protrusions 30.

In the example shown, the variable locating dimensions 34 may correspond to at least one of a first longitudinal position of the fore aperture 36a, a first length of the fore aperture 36a, and a second length of the aft apertures 36b. For example, each of the dimensions associated with the corresponding second length of the aft aperture 36b, the first length of the fore aperture 36a, and the center or longitudinal position of the fore aperture 36a may vary in response to an adjustment in the thickness T of the blade 12 as exemplified in Tables 1 and 2. As shown, the thicknesses T of the exemplary blades 12 associated with the blade apparatus discussed herein may vary from approximately 0.25 mm to 2 mm. Accordingly, the variable locating dimensions 34 of the blade apparatus 12 may ensure that the features of the locking interface 160, for example, the first tapered opposing sides 154, second tapered opposing sides 156, and/or the ramp portion 140 may consistently engage the blades 12 having differing blade thicknesses T. In addition to the variable locating dimensions 34, at least one of the dimensions, in this case the fore center or a second longitudinal position of the aft aperture 36e, may be fixed or constant over the range of thicknesses T for the blades of the blade apparatus 12.

TABLE 1
Example Blade Dimensions - Aft Mounting Aperture

| Thickness | | Aft | | Aft | |
|---|---|---|---|---|---|
| (T) | DIM A | Center | DIM B | Length (L) | Ratio T/L |
| 0.250 | 0.198 | 0.256 | 0.314 | 0.116 | 2.151 |
| 0.380 | 0.196 | 0.256 | 0.316 | 0.119 | 3.188 |
| 0.600 | 0.196 | 0.256 | 0.316 | 0.121 | 4.967 |
| 0.800 | 0.195 | 0.256 | 0.317 | 0.122 | 6.547 |
| 0.900 | 0.195 | 0.256 | 0.317 | 0.123 | 7.329 |
| 1.000 | 0.194 | 0.256 | 0.318 | 0.124 | 8.091 |
| 1.190 | 0.194 | 0.256 | 0.318 | 0.125 | 9.535 |
| 1.200 | 0.194 | 0.256 | 0.318 | 0.125 | 9.615 |
| 1.270 | 0.193 | 0.256 | 0.319 | 0.125 | 10.128 |
| 1.370 | 0.193 | 0.256 | 0.319 | 0.126 | 10.873 |
| 1.470 | 0.193 | 0.256 | 0.319 | 0.127 | 11.593 |
| 1.820 | 0.192 | 0.256 | 0.320 | 0.128 | 14.208 |
| 1.970 | 0.192 | 0.256 | 0.321 | 0.129 | 15.248 |

Dimensions in millimeters

Though discussed in reference to exemplary variable locating dimensions 34 identified in Tables 1 and 2, various aspects of the dimensions of the apertures 36 forming the mounting interface 22 may generally vary to compensate for changes in proportions of one or more tapered portions or surfaces formed by the protrusions 30. In the specific example demonstrated, the thicknesses T may range from 0.2 mm to 2 mm, in conjunction with the proportions of each of the apertures 36, which may be defined as a thickness-to-length ratio. As further demonstrated in Table 1, a thickness-to-length ratio of the second length or aft length of the aft aperture 36b may vary from approximately 2.2 to 15.3 over the range of thickness from 2.5 mm to 2 mm.

TABLE 2
Example Blade Dimensions - Fore Mounting Aperture

| Thickness | | Fore | | Fore | Ratio |
|---|---|---|---|---|---|
| (T) | DIM C | Center | DIM D | Length (L) | (T/L) |
| 0.250 | 0.450 | 0.567 | 0.685 | 0.235 | 1.063 |
| 0.380 | 0.449 | 0.571 | 0.692 | 0.242 | 1.568 |
| 0.600 | 0.449 | 0.583 | 0.717 | 0.268 | 2.235 |
| 0.800 | 0.448 | 0.593 | 0.739 | 0.291 | 2.748 |
| 0.900 | 0.448 | 0.598 | 0.748 | 0.301 | 2.994 |
| 1.000 | 0.447 | 0.604 | 0.761 | 0.314 | 3.187 |
| 1.190 | 0.447 | 0.613 | 0.780 | 0.333 | 3.571 |
| 1.200 | 0.447 | 0.613 | 0.780 | 0.333 | 3.601 |
| 1.270 | 0.446 | 0.618 | 0.789 | 0.343 | 3.704 |
| 1.370 | 0.446 | 0.622 | 0.799 | 0.353 | 3.884 |
| 1.470 | 0.446 | 0.628 | 0.811 | 0.366 | 4.021 |
| 1.820 | 0.446 | 0.256 | 0.826 | 0.380 | 4.796 |
| 1.970 | 0.445 | 0.256 | 0.840 | 0.395 | 4.991 |

Dimensions in millimeters

As demonstrated in Table 2, the thickness-to-length ratio of the first length or fore length of the fore aperture 36a may range from 1 to 5 over the range of thickness from 0.25 mm to 2 mm. In various implementations as further discussed in reference to FIGS. 7A, 7B, and 7C, the second length or aft length of the aft aperture 36b may be less than the first length or fore length of the fore aperture 36a. In this configuration, the second length or aft length of the aft aperture 36b may be insufficient to receive the fore protrusion 30a. In this way, the aft aperture 36b may freely slide over the fore protrusion 30a unencumbered in order to reach the aft protrusion 30b, which may be deeper within the blade-receiving opening 20 or further along the blade-receiving axis A_{B} than the fore protrusion 30a.

In addition to the dimensions of the apertures 36, one or more features may be incorporated in the apertures 36 to further improve the mounting and engagement of the locking interface 160 to the mounting interface 22. As demonstrated in FIG. 6B, the fore aperture 36a may correspond to a rectangular shape with straight sidewalls. In contrast, the aft aperture 36b may comprise shaped opposing ends 170, exemplified as rounded ends, laterally spaced over the blade width W across the longitudinal axis A_{L}. In this configuration, the aft aperture 36b and the aft protrusion 30b may form a different distal profile geometry than the fore protrusion 30a and the fore aperture 36a that is incompatible for mating along one or more sides of the profile shapes. For example, the rectangular profile of the fore protrusion 30a may be incompatible with the rounded profile of the aft aperture 36b to prevent binding or interference when mounting the blade 12 along the blade-receiving axis A_{B}.

Though referred to as shaped opposing ends 170 and particularly demonstrated as round ends, the opposing walls of the aft aperture 36b may be reduced in a variety of shapes within a mating profile formed by the fore protrusion 30a. For example, one or more sides or corners of the aft aperture 36b may be fileted, chamfered, tapered, or otherwise shaped from a fore wall 172, an aft wall 174, or evenly from each wall 172, 174. In this configuration, the shaped opposing ends 170 may assist in ensuring that the aft aperture 36b easily slides over the fore protrusion 30a and through the clearance region C without catching on the fore protrusion 30a. Additionally, the shaped opposing ends 170 may engage corresponding shaped ends 180 of the aft protrusion 30b to improve the engagement of the locking interface 160 to the mounting interface 22.

Referring now to FIGS. 7A, 7B, 7C, and 7D, a method of mounting the blade 12 to the mounting apparatus 10 is described in further detail. As demonstrated in FIG. 7A, the method may begin by guiding the second end portion 12b of the blade 12 comprising the mounting interface 22 into the receiving opening 20 by aligning the longitudinal axis A_{L} of the blade 12 with the blade-receiving axis A_{B}. As shown, the mating profile 130 of the blade 12 may extend between the opposing slots 124 formed in the opposing sides 122 and compress the spring in connection with the locking detent 18 along the rotational axis A_{R} by engaging the ramp portion 140 and a top interior wall of each of the opposing slots 124. Once inserted, the clearance region C may form or expand within the receiving opening 20, such that the thickness T of the blade 12 may slide along the ramp portion 140, over the elongated protrusion surface 144, and beyond the transverse recess 146 as demonstrated in FIG. 7B.

As the second end portion 12b of the blade 12 engages the blade-receiving opening 20, the aft aperture 36b may be guided over and beyond the fore protrusion 30a. As previously discussed, one or more receiving dimensions, for example, the length, width, and/or shape of the aft aperture 36b, may be proportioned such that the aft aperture 36b is insufficient and/or incompatible to receive the shape and/or proportions of the fore protrusion 30a. In this configuration, the aft aperture 36b may easily slide over the mating surfaces of the fore protrusion 30a. As demonstrated in FIG. 7B, the blade 12 may further comprise an intermediate portion 12c which may further be guided over the ramp portion 140 and the elongated protrusion surface 144 of the fore protrusion 30a until the second end portion 12b of the blade 12 is at least partially aligned with the aft protrusion 30b along the mating axis A_{M} and within the receiving opening 20. The intermediate portion 12c of the blade 12 may be positioned between and partially define the fore aperture 36a and the aft aperture 36b. In this configuration, the intermediate portion 12c may engage a portion of the fore protrusion 30a during the loading operation to maintain the clearance region C for the passage of the second end portion 12b of the blade 12 to extend within the receiving opening 20 beyond the transverse recess 146. In this way, the interaction between the mounting interface 22 and the locking interface 160 may prevent the blade 12 from binding within the receiving opening 20 during the loading operation as discussed herein.

Referring now to FIG. 7C, the fore aperture 36a and the aft aperture 36b may align concurrently with the fore protrusion 30a and the aft protrusion 30b. In this configuration, the second end portion 12b of the blade 12 may be positioned between the release tab 26 and the aft protrusion 30b. Additionally, the intermediate portion 12c of the blade 12 may be positioned between the fore protrusion 30a and the aft protrusion 30b within the transverse recess 146 of the locking detent 18. Finally, the extent of the blade 12 extending from the fore aperture 36a to the first end portion 12a or acting end may be positioned along the ramp portion 140 of the locking detent 18. In this aligned mating position, the locking detent 18 may be extended into the clearance portion C as a result of the alignment of the protrusions 30 with the corresponding apertures 36. In this configuration, the protrusions 30 may align within the apertures 36, such that the tapered opposing sides 154, 156 align and bind with the corresponding mounting surfaces within the apertures 36. As previously discussed, the relationship of the proportions and/or positions of the variable locating dimensions 34 of the apertures 36 may ensure that blades 12 having various thicknesses T securely mount within the receiving opening 20 as a result of the engagement of the protrusions 30 with the corresponding apertures 36.

As demonstrated in FIG. 7D, the blade 12 may be unloaded from the mounting apparatus 10 via a simple operation initiated by applying a compression force, denoted as reference A, downward toward a mounting apparatus 10 along the mating axis A_{M}. The compression force applied to the release tab 26 may result in the compression of the spring mechanism 112, thereby allowing the locking detent 18 to translate out of the clearance region C. With the locking detent 18 disengaged from the blade 12, the blade 12 may be withdrawn along the longitudinal axis A_{L} outward through the receiving opening 20 in an ejection direction denoted as reference B. In this way, the blade-mounting apparatus 10 may provide for the secure mounting of the blade 12 to the surgical tool 14 while ensuring that blades having a variety of thicknesses T may be secured and accommodated for use.

According to some aspects of the disclosure, a blade-mounting apparatus for a surgical tool comprises an attachment head including a mounting channel with a plurality of opposing sides and a base, and opposing slots formed in each of the opposing sides and aligned across the mounting channel forming a blade-receiving opening extending therebetween and aligned with a receiving axis. A locking detent is disposed within the mounting channel and has an engagement surface comprising a plurality of protrusions including a first protrusion proximal to the receiving opening and a second protrusion distal to the receiving opening, wherein the second protrusion is elongated transverse to the receiving axis and tapered from the engagement surface to a distal extent.

According to various aspects, the disclosure may implement one or more of the following features or configurations in various combinations:
the second protrusion is configured to engage an elongated slot of the blade extending transverse to a longitudinal axis of the blade;
the receiving opening receives a longitudinal axis of the blade aligned with the receiving axis in a blade-loading operation;
the protrusions form a transverse recess extending between the opposing sides and tapering from an extent of the protrusions to the engagement surface;
the second protrusion further comprises rounded ends on opposing sides laterally spaced from the receiving axis;
the locking detent further comprises a release tab extending from the engagement surface;
the release tab is positioned opposite the receiving opening and forms a back surface within the blade-receiving opening in a loading configuration, wherein the protrusions of the locking detent are retracted from the blade-receiving opening;
the opposing slots extend unencumbered from a receiving end to an open terminal end of the opposing sides, wherein the receiving end receives the blade into the receiving opening;
a detent shaft extending from a connection surface of the locking detent opposite the engagement surface;
the detent shaft extends centrally through the base of the attachment head along an actuation axis about which the blade is rotated by the surgical tool;
the attachment head comprises a shank extending from the base and connected to a drive link of a drive head of the surgical tool, wherein the shank and the attachment head are driven by the drive link of the surgical tool about the actuation axis;
the shank forms a drive coupler and the drive link forms a drive socket configured to receive the drive head;
the drive coupler is positioned along an intermediate portion of the shank disposed between the base of the attachment head and a distal end portion of the shank;
the detent shaft forms a stem that extends through a bore of the shank to a retaining end;
a spring disposed in the bore between the shank of the attachment head and the detent shaft connected to the locking detent;
the retaining end comprises an annular groove configured to receive a retaining ring to secure the detent shaft within the bore of the shank; and/or
the detent shaft extends to a locking configuration wherein the spring is extended and the protrusions engage the blade within the receiving opening; and in response to a compression of the spring, the detent shaft is retracted into the shank of the attachment head in a loading configuration, wherein the protrusions of the locking detent are retracted from the blade-receiving opening.

According to another aspect of the disclosure, a surgical blade apparatus has a thickness that varies over a range of blade thicknesses for mating with a compatible receiving opening of a blade attachment assembly of a surgical tool. The surgical blade apparatus comprises an elongated rectangular mating profile defining a blade width and a blade thickness extending along a longitudinal axis of the surgical blade apparatus configured to engage the blade-receiving opening, an acting end disposed at a first end portion, and a mounting interface disposed at a second end portion opposite the first end portion. The mounting interface comprises a plurality of mating apertures configured to receive a plurality of mating protrusions formed through the surgical blade along a mating axis perpendicular to the longitudinal axis. The plurality of apertures comprises a fore aperture formed at a first longitudinal position comprising a first length and a first width and forming a first receiving perimeter wall configured to receive the first protrusion of the mating protrusions and an aft aperture formed at a second longitudinal position comprising a second length and a second width and forming a second receiving perimeter wall configured to receive the second protrusion of the mating protrusions.

According to various aspects, the disclosure may implement one or more of the following features or configurations in various combinations:
the fore aperture and the aft aperture comprise a plurality of variable locating dimensions defined by at least one of the first longitudinal position, the first length, and the second length;
at least one of the plurality of variable locating dimensions is positioned along the longitudinal axis of the blade in response to the blade thickness;
the variable locating dimensions are defined by the blade thickness over the range of blade thicknesses and align with the mating protrusions over the range of blade thicknesses;
the second longitudinal position of the aft aperture is fixed over the range of blade thicknesses;
the first longitudinal position of the fore aperture of the blade is changed in response to the blade thickness;
at least one of the first length and the second length vary in response to the blade thickness;
the range of blade thicknesses is from 0.25 mm-2 mm;
a fore aperture ratio of the first length to the blade thickness ranges from 2.2 to 15.3 over the range of blade thicknesses;
an aft aperture ratio of the second length to the blade thickness ranges from 1 to 5 over the range of blade thicknesses;
the second length is less than the first length and the first width;
the mating protrusions comprise a fore protrusion and an aft protrusion and the aft aperture engages the mating protrusions before the fore aperture in a loading operation;
the second length is insufficient to receive the fore protrusion, thereby allowing the aft protrusion to slide over the fore protrusion and receive the aft protrusion in the loading operation;
the aft aperture is elongated transverse to the longitudinal axis; and/or
the aft aperture further comprises rounded ends on opposing sides laterally spaced from the receiving axis.

According to yet another aspect of the disclosure, a method of mounting a blade to a blade-mounting apparatus of a surgical tool comprises guiding a first end of the blade along a receiving axis of a receiving opening formed between opposing slots aligned over a mounting channel; sliding the first end of the blade along a ramp of a fore protrusion of a locking detent by displacing the locking detent from a clearance portion of the receiving opening; guiding an aft aperture of the blade over and beyond the fore protrusion, wherein a receiving dimension of the aft aperture of the blade is insufficient to receive the fore protrusion; aligning a fore aperture of the blade over the fore protrusion concurrently with aligning the aft aperture of the blade over an aft protrusion of the locking detent; and locking the blade into a mounted position by extending the locking detent into the clearance portion of the receiving opening, thereby engaging the fore protrusion in the fore aperture and the aft protrusion in the aft aperture.

According to various aspects, the disclosure may implement one or more of the following features or configurations in various combinations:
guiding an intermediate blade portion disposed between the aft aperture and a fore aperture of the blade along the receiving axis over the fore protrusion until the first end of the blade is at least partially aligned with an aft protrusion of the locking detent, thereby maintaining the clearance portion of the receiving opening;
the locking detent is displaced by compressing a spring configured to extend the locking detent into the clearance portion;
the locking detent is extended into the clearance portion of the receiving opening by a force of the spring; and/or
releasing the blade from the locking detent by compressing the spring via a compression force applied to a release tab extending from the locking detent.

It will be understood that any described processes or steps within described processes may be combined with other disclosed processes or steps to form structures within the scope of the present device. The exemplary structures and processes disclosed herein are for illustrative purposes and are not to be construed as limiting.

It is also to be understood that variations and modifications can be made on the aforementioned structures and methods without departing from the concepts of the present device, and further it is to be understood that such concepts are intended to be covered by the following claims unless these claims by their language expressly state otherwise.

The above description is considered that of the illustrated embodiments only. Modifications of the device will occur to those skilled in the art and to those who make or use the device. Therefore, it is understood that the embodiments shown in the drawings and described above are merely for illustrative purposes and not intended to limit the scope of the device, which is defined by the following claims as interpreted according to the principles of patent law, including the Doctrine of Equivalents

1. A blade-mounting apparatus for a surgical tool comprising:
an attachment head comprising a mounting channel comprising a plurality of opposing sides and a base, and opposing slots formed in each of the opposing sides and aligned across the mounting channel forming a blade-receiving opening extending therebetween and aligned with a receiving axis; and
a locking detent disposed within the mounting channel and having an engagement surface comprising a plurality of protrusions including a first protrusion proximal to the receiving opening and a second protrusion distal to the receiving opening, wherein the second protrusion is elongated transverse to the receiving axis and tapered from the engagement surface to a distal extent.

2. The apparatus according to claim 1, wherein the second protrusion is configured to engage an elongated slot of the blade extending transverse to a longitudinal axis of the blade.

3. The apparatus according to claim 1, wherein the receiving opening receives a longitudinal axis of the blade aligned with the receiving axis in a blade-loading operation.

4. The apparatus according to claim 1, wherein the protrusions form a transverse recess extending between the opposing sides and tapering from an extent of the protrusions to the engagement surface.

5. The apparatus according to claim 1, wherein the second protrusion further comprises rounded ends on opposing sides laterally spaced from the receiving axis.

6. The apparatus according to claim 1, wherein the locking detent further comprises a release tab extending from the engagement surface.

7. The apparatus according to claim 1, wherein the release tab is positioned opposite the receiving opening and forms a back surface within the blade-receiving opening in a loading configuration, wherein the protrusions of the locking detent are retracted from the blade-receiving opening.

8. The apparatus according to claim 1, wherein the opposing slots extend unencumbered from a receiving end to an open terminal end of the opposing sides, wherein the receiving end receives the blade into the receiving opening.

9. The apparatus according to claim 1, further comprising:
a detent shaft extending from a connection surface of the locking detent opposite the engagement surface, wherein the detent shaft extends centrally through the base of the attachment head along an actuation axis about which the blade is rotated by the surgical tool.

10. A surgical blade apparatus with a thickness that varies over a range of blade thicknesses for mating with a compatible receiving opening of a blade attachment assembly of a surgical tool, the surgical blade apparatus comprising:
an elongated rectangular mating profile defining a blade width and a blade thickness extending along a longitudinal axis of the surgical blade apparatus configured to engage the blade-receiving opening;
an acting end disposed at a first end portion; and
a mounting interface disposed at a second end portion opposite the first end portion, wherein the mounting interface comprises a plurality of mating apertures configured to receive a plurality of mating protrusions formed through the surgical blade along a mating axis perpendicular to the longitudinal axis, wherein the plurality of apertures comprises:
a fore aperture formed at a first longitudinal position comprising a first length and a first width and forming a first receiving perimeter wall configured to receive the first protrusion of the mating protrusions; and
an aft aperture formed at a second longitudinal position comprising a second length and a second width and forming a second receiving perimeter wall configured to receive the second protrusion of the mating protrusions.

11. The surgical blade apparatus according to claim 10, wherein the fore aperture and the aft aperture comprise a plurality of variable locating dimensions defined by at least one of the first longitudinal position, the first length, and the second length.

12. The surgical blade apparatus according to claim 11, wherein at least one of the plurality of variable locating dimensions is positioned along the longitudinal axis of the blade in response to the blade thickness.

13. The surgical blade apparatus according to claim 11, wherein the variable locating dimensions are defined by the blade thickness over the range of blade thicknesses and align with the mating protrusions over the range of blade thicknesses.

14. The surgical blade apparatus according to claim 11, wherein the second longitudinal position of the aft aperture is fixed over the range of blade thicknesses.

15. The surgical blade apparatus according to claim 14, wherein the first longitudinal position of the fore aperture of the blade is changed in response to the blade thickness.

16. The surgical blade apparatus according to claim 14, wherein at least one of the first length and the second length vary in response to the blade thickness.

17. The surgical blade apparatus according to claim 11, wherein the range of blade thicknesses is from 0.25 mm-2 mm.

18. The surgical blade apparatus according to claim 17, wherein a fore aperture ratio of the first length to the blade thickness ranges from 2.2 to 15.3 over the range of blade thicknesses.

19. The surgical blade apparatus according to claim 17, wherein an aft aperture ratio of the second length to the blade thickness ranges from 1 to 5 over the range of blade thicknesses.

20. A method of mounting a blade to a blade-mounting apparatus of a surgical tool comprising:
guiding a first end of the blade along a receiving axis of a receiving opening formed between opposing slots aligned over a mounting channel;
sliding the first end of the blade along a ramp of a fore protrusion of a locking detent by displacing the locking detent from a clearance portion of the receiving opening;
guiding an aft aperture of the blade over and beyond the fore protrusion, wherein a receiving dimension of the aft aperture of the blade is insufficient to receive the fore protrusion;
aligning a fore aperture of the blade over the fore protrusion concurrently with aligning the aft aperture of the blade over an aft protrusion of the locking detent; and
locking the blade into a mounted position by extending the locking detent into the clearance portion of the receiving opening, thereby engaging the fore protrusion in the fore aperture and the aft protrusion in the aft aperture.
